(19) United States
(12) Patent Application Publication
(10) Pub. No.: US 2023/0103344 A1
(43) Pub. Date: Apr. 6, 2023

(54) X-Ray Imaging Method and X-Ray Imaging System

(71) Applicant: SHIMADZU CORPORATION, Kyoto-shi (JP)

(72) Inventor: Hisanori MORITA, Kyoto-shi (JP)

(21) Appl. No.: 17/635,648

(22) PCT Filed: Aug. 21, 2019
(86) PCT No.: PCT/JP2019/032633

§ 371 Date: Jun. 10, 2022

(51) Int. Cl. A61B 6/00; G06T 3/40; G06T 7/00

(52) U.S. Cl. CPC A61B 6/542; G06T 3/4046; G06T 7/0012; G06T 2207/10116; G06T 2207/20081

(57) ABSTRACT

An X-ray imaging method of taking an X-ray image of a subject includes irradiating the subject with an X-ray at a first dose and taking a first X-ray image of the subject, irradiating the subject with an X-ray at a second dose lower than the first dose and taking a second X-ray image of the subject, and inputting the second X-ray image into a trained model trained by machine learning to modify the second X-ray image.

### TECHNICAL FIELD

The present invention relates to an X-ray imaging method and an X-ray imaging system.

### BACKGROUND ART

Japanese Patent Laying-Open No. 2018-46905 (PTL 1) discloses a radiography apparatus that takes a fluoroscopic image which is imaging of the inside of a subject by irradiating the subject with an X-ray. This radiography apparatus successively generates fluoroscopic images by intermittently emitting an X-ray at a prescribed time interval, and shows the images on a monitor in a format of moving images.

### Citation List

### Patent Literature

PTL 1: Japanese Patent Laying-Open No. 2018-46905

### SUMMARY OF INVENTION

### Technical Problem

X-ray images (moving images) to be used for a medical purpose are required to be high in image quality. In order to obtain high image quality, a subject is desirably irradiated with an X-ray intermittently at a short time interval and at a high dose. On the other hand, dosage of the subject in X-ray imaging is desirably minimized.

A longer time interval for irradiation with the X-ray for lowering the dosage of a subject, however, leads to lowering in frame rate. In other words, since an interval between frames becomes longer, information between frames may be missed. In addition, lowering in dose of the X-ray for lowering the dosage of the subject may lead to lowering in quality of an X-ray image and an unclear X-ray image. In other words, there is a trade-off between lowering in dosage of the subject in X-ray imaging and improvement in image quality of an X-ray image.

The present invention was made to solve the problem above, and an object thereof is to lower dosage of a subject while lowering in image quality of an X-ray image is suppressed.

### Solution to Problem

A first aspect of the present invention is directed to an X-ray imaging method of taking an X-ray image of a subject, and the X-ray imaging method includes irradiating the subject with an X-ray at a first dose and taking a first X-ray image of the subject, irradiating the subject with an X-ray at a second dose lower than the first dose and taking a second X-ray image of the subject, and inputting the second X-ray image into a trained model trained by machine learning to modify the second X-ray image.

A second aspect of the present invention is directed to an X-ray imaging method of taking an X-ray image of a subject, and the X-ray imaging method includes irradiating the subject with an X-ray at a prescribed time interval and taking a third X-ray image and a fourth X-ray image of the subject that are successive and generating an intermediate image between the third X-ray image and the fourth X-ray image by using the third X-ray image and the fourth X-ray image.

A third aspect of the present invention is directed to an X-ray imaging method of taking an X-ray image of a subject, and the X-ray imaging method includes irradiating the subject with an X-ray and generating an X-ray image of the subject and generating a prediction image in a next frame of the X-ray image by using the generated X-ray image.

A fourth aspect of the present invention is directed to an X-ray imaging system including an imaging apparatus configured to successively generate X-ray images of a subject by irradiating the subject with an X-ray and an image processing apparatus that processes the X-ray images. The imaging apparatus is configured to perform processing for irradiating the subject with an X-ray at a first dose and taking a first X-ray image of the subject and processing for irradiating the subject with an X-ray at a second dose lower than the first dose and taking a second X-ray image of the subject. The image processing apparatus is configured to input the second X-ray image into a trained model trained by machine learning to modify the second X-ray image.

A fifth aspect of the present invention is directed to an X-ray imaging system including an imaging apparatus and an image processing apparatus. The imaging apparatus is configured to take a third X-ray image and a fourth X-ray image of a subject that are successive, by irradiating the subject with an X-ray at a prescribed time interval. The image processing apparatus is configured to generate an intermediate image intermediate between the third X-ray image and the fourth X-ray image by using the third X-ray image and the fourth X-ray image.

A sixth aspect of the present invention is directed to an X-ray imaging system including an imaging apparatus configured to successively generate X-ray images of a subject by irradiating the subject with an X-ray and an image processing apparatus configured to generate a prediction image in a next frame of the X-ray images by using the generated X-ray images.

### Advantageous Effects of Invention

According to the present invention, dosage of a subject can be lowered while lowering in image quality of an X-ray image is suppressed.

### BRIEF DESCRIPTION OF DRAWINGS

FIG. 1 is a diagram of an overall configuration of an X-ray imaging system according to a first embodiment.

FIG. 2 is a diagram schematically showing a construction of a catheter used in a coronary artery (cardiovascular) intervention therapy by using the X-ray imaging system.

FIG. 3 is a schematic diagram for illustrating taking of X-ray moving images according to the first embodiment.

FIG. 4 is a diagram for illustrating an exemplary first X-ray image.

FIG. 5 is a diagram for illustrating an exemplary second X-ray image.

FIG. 6 is a flowchart showing an exemplary processing procedure performed in an imaging apparatus and an image processing apparatus according to the first embodiment.

FIG. 7 is a flowchart showing an exemplary processing procedure performed in the imaging apparatus and the image processing apparatus according to a first modification.

FIG. 8 is a schematic diagram for illustrating taking of X-ray moving images according to a second modification.

FIG. 9 is a flowchart showing an exemplary processing procedure performed in the imaging apparatus and the image processing apparatus according to the second modification.

FIG. 10 is a schematic diagram for illustrating taking of X-ray moving images according to a second embodiment.

FIG. 11 is a flowchart showing an exemplary processing procedure performed in the imaging apparatus and an image processing apparatus according to the second embodiment.

FIG. 12 is a schematic diagram for illustrating taking of X-ray moving images according to a fifth modification.

FIG. 13 is a flowchart showing an exemplary processing procedure performed in the imaging apparatus and the image processing apparatus according to the fifth modification.

FIG. 14 is a schematic diagram for illustrating taking of X-ray moving images according to a third embodiment.

FIG. 15 is a flowchart showing an exemplary processing procedure performed in the imaging apparatus and an image processing apparatus according to the third embodiment.

### DESCRIPTION OF EMBODIMENTS

An embodiment of the present invention will be described in detail below with reference to the drawings. The same or corresponding elements in the drawings have the same reference characters allotted and description thereof will not be repeated.

### First Embodiment

### Overall Configuration

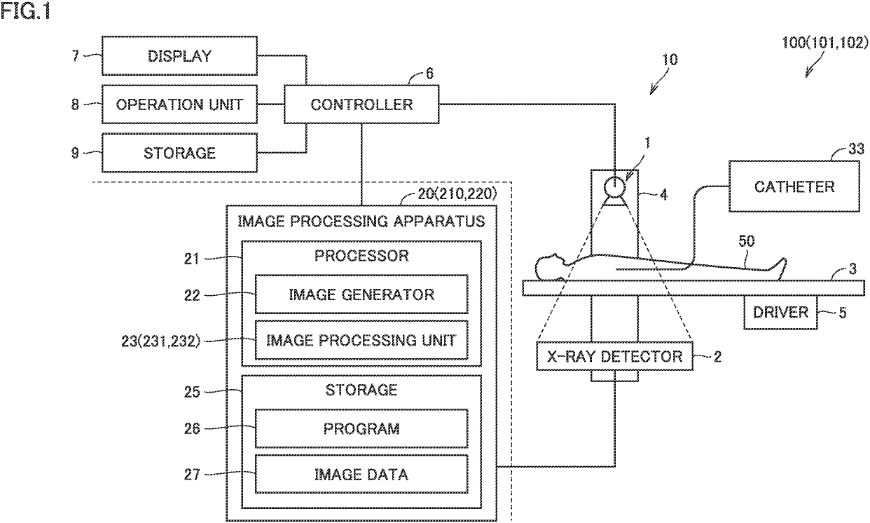

FIG. 1 is a diagram of an overall configuration of an X-ray imaging system 100 according to a first embodiment. X-ray imaging system 100 takes an X-ray image which is imaging of the inside of a subject 50 such as a human body, by irradiation of subject 50 with an X-ray. Referring to FIG. 1, X-ray imaging system 100 includes an imaging apparatus 10 and an image processing apparatus 20.

Imaging apparatus 10 includes an X-ray emitter 1, an X-ray detector 2, an imaging table 3, a movement mechanism 4, a driver 5, a controller 6, a display 7, an operation unit 8, and a storage 9.

X-ray emitter 1 includes an X-ray tube and a collimator (neither of which is shown). The X-ray tube is connected to a high voltage generator and it generates an X-ray by application of a high voltage thereto. The collimator is provided in the X-ray tube and adjusts a field of irradiation with the X-ray emitted from the X-ray tube. X-ray emitter 1 generates an X-ray in accordance with an imaging condition set by controller 6. The condition for imaging includes, for example, a tube voltage, a tube current, and a time interval or a pulse width in irradiation with the X-ray.

X-ray detector 2 is arranged as being opposed to X-ray emitter 1 with imaging table 3 being interposed. X-ray detector 2 detects the X-ray that is emitted from X-ray emitter 1 and has passed through subject 50 and imaging table 3. Then, X-ray detector 2 provides a detection signal in accordance with intensity of the detected X-ray to image processing apparatus 20. X-ray detector 2 is representatively implemented by a flat panel detector (which is also referred to as an “FPD” below).

X-ray emitter 1 and X-ray detector 2 are movably supported by movement mechanism 4. Imaging table 3 is movable by driver 5. By moving X-ray emitter 1, X-ray detector 2, and imaging table 3, an imaging area in subject 50 to be imaged can be moved.

Controller 6 includes a central processing unit (CPU), a memory (a read only memory (ROM) and a random access memory (RAM)), and an input and output buffer for input and output of various signals (none of which is shown). Controller 6 controls each component of imaging apparatus 10 and image processing apparatus 20 by executing a control program based on provided various signals or the like. Controller 6 controls each component and image processing apparatus 20 for performing first processing and second processing which will be described later in X-ray imaging system 100.

Display 7 is a monitor such as a liquid crystal display. Display 7 shows an X-ray image generated by image processing apparatus 20 or an X-ray image stored in storage 9 in accordance with an instruction from controller 6.

Operation unit 8 is an input device operable by a doctor or a technologist who uses X-ray imaging system 100 (who is also simply referred to as a “user” below). With the use of operation unit 8, for example, the user can give an instruction to start/quit X-ray imaging with X-ray imaging system 100, set an imaging condition for X-ray imaging system 100, or indicate a state of display on display 7.

Storage 9 includes a storage of a high capacity such as a hard disk drive or a solid state drive. Image data of X-ray images shown on display 7 is stored in storage 9 for reproduction after end of imaging by X-ray imaging system 100.

Image processing apparatus 20 includes a processor 21 and a storage 25. An image processing program for performing various types of image processing is stored in storage 25. Functions of an image generator 22 and an image processing unit 23 are performed by execution of the image processing program by processor 21. Each of image generator 22 and image processing unit 23 may be implemented by a dedicated processor.

Image generator 22 generates an X-ray image based on a detection signal obtained from X-ray detector 2. Image generator 22 according to the first embodiment successively generates X-ray images in a format of moving images based on detection signals successively provided from X-ray detector 2. Specifically, X-ray emitter 1 intermittently emits X-rays to subject 50 at a prescribed time interval. Then, X-ray detector 2 successively detects X-rays that have passed through subject 50. Image generator 22 successively generates X-ray images at a prescribed frame rate by generating X-ray images based on detection signals successively obtained from X-ray detector 2. The frame rate is, for example, approximately from 15 FPS to 30 FPS.

Image processing unit 23 is configured to perform image processing (third processing which will be described later) on an X-ray image generated by image processing unit 22.

Image processing apparatus 20 provides an X-ray image generated by image generator 22 and an X-ray image subjected to image processing by image processing unit 23 to imaging apparatus 10. Controller 6 of imaging apparatus 10 can have the X-ray image of subject 50 shown in real time by having display 7 show the X-ray image obtained from image processing apparatus 20. Image processing apparatus 20 may store an X-ray image generated by image generator 22 and/or an X-ray image subjected to image processing by image processing unit 23 in storage 25 as image data 27.

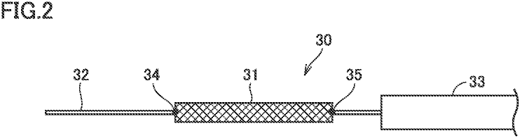

FIG. 2 is a diagram schematically showing a construction of a catheter used in a coronary artery (cardiovascular) intervention therapy by using X-ray imaging system 100. Referring to FIGS. 1 and 2, a catheter 33 contains a guide wire 32. A stent 31 is provided in guide wire 32. Stent 31 is constructed in a cylindrical shape with a network structure formed, for example, of a metal or a resin. Markers 34 and 35 for specifying a position of stent 31 during X-ray imaging are provided at opposing ends of stent 31. Markers 34 and 35 are members through which an X-ray does not pass, the markers being composed of a metal such as gold, platinum, or tantalum. By sensing the positions of markers 34 and 35 in a taken X-ray image, the position of stent 31 can be specified.

In the coronary artery intervention therapy, catheter 33 is inserted in a blood vessel of subject 50 to reach the coronary artery of the heart. Then, stent 31 is disposed in a narrowed part of the blood vessel and inflated by a balloon (not shown) provided therein, so that stent 31 indwells. The narrowed part is thus expanded to keep bloodstream normal.

### Taking of X-Ray Moving Images

In the coronary artery intervention therapy as above, the position or the like of stent 31 should accurately be known. Therefore, the X-ray image should be high in image quality. In order to obtain high image quality (moving image quality) in taking X-ray images (moving images), desirably, X-ray images are generated by intermittent emission of X-rays at a short time interval and at a high dose. As the dose of the X-rays is higher, quality of generated X-ray images (that is, X-ray moving images) is higher, and as a time interval of emission of the X-rays is shorter, moving images that follow actual operations can be taken.

On the other hand, lowering in dosage of subject 50 in X-ray imaging is desired. A longer time interval for irradiation with the X-ray for lowering dosage of subject 50 leads to lowering in frame rate and a longer interval between frames. Then, information between frames may be missed. Lowering in dose of the X-ray for lowering dosage of subject 50 may lead to lowering in quality of an individual X-ray image and an unclear X-ray image. In other words, there is a trade-off between lowering in dosage of subject 50 in X-ray imaging and improvement in moving image quality of X-ray moving images.

Then, in the first embodiment, in X-ray imaging system 100 that generates an X-ray image by irradiating subject 50 with the X-ray at a prescribed time interval, a dose of the X-ray emitted to subject 50 is lowered every other time. Dosage of subject 50 in taking X-ray moving images can thus be lowered. An X-ray image generated with the dose of the X-ray being lowered is lower in quality than an X-ray image generated without lowering in dose of the X-ray. In order to address this, processing for improving the quality of the X-ray image generated with the dose of the X-ray being lowered is further performed. Thus, even when the dose of the X-ray emitted to subject 50 is lowered every other time, lowering in moving image quality of X-ray moving images can be suppressed.

The dose of the X-ray can be adjusted by varying the tube current, or the time interval or the pulse width of irradiation with the X-ray. Instead of adjustment of the dose of the X-ray, emission energy may be adjusted by varying the tube voltage (a skin dose is thus also varied).

Instead of adjusting the dose of the X-ray, an area of irradiation with the X-ray may be adjusted. The area of irradiation with the X-ray can be adjusted by varying an aperture of the collimator or by inserting and removing a shield with a hole.

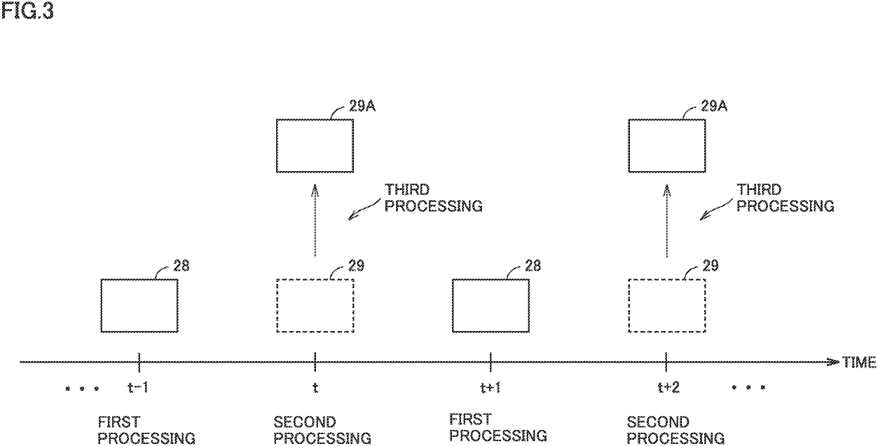

FIG. 3 is a schematic diagram for illustrating taking of X-ray moving images according to the first embodiment. FIG. 3 shows irradiation with the X-ray at a prescribed time interval (..., t-1, t, t+1, t+2, ...) and generation of an X-ray image at each time point.

At time t-1 and time t+1, first processing for irradiating subject 50 with the X-ray at a first dose to generate an X-ray image (which is also referred to as a “first X-ray image” below) 28 of subject 50 is performed. At time t and time t+2, second processing for irradiating subject 50 with the X-ray at a second dose lower than the first dose to generate an X-ray image (which is also referred to as a “second X-ray image” below) 29 of subject 50 is performed. In other words, the first processing and the second processing are alternately performed at a prescribed time interval.

By alternately performing the first processing and the second processing at the prescribed time interval as above, dosage of subject 50 can be lower than in an example where the first processing is performed at the prescribed time interval.

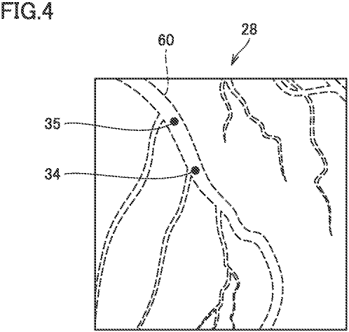

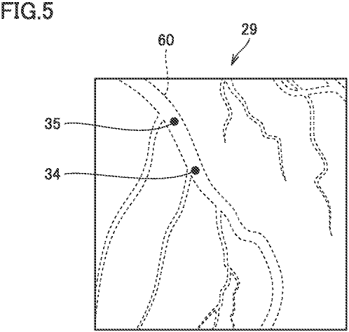

Second X-ray image 29 generated with the dose of the X-ray being lowered, however, is lower in quality than first X-ray image 28. FIGS. 4 and 5 show one example. FIG. 4 is a diagram for illustrating exemplary first X-ray image 28. FIG. 5 is a diagram for illustrating exemplary second X-ray image 29. FIGS. 4 and 5 each show an X-ray image generated in the coronary artery intervention therapy. As can be recognized by comparing FIGS. 4 and 5 with each other, second X-ray image 29 generated at the dose of the X-ray lower than the dose for first X-ray image 28 is lower in quality than first X-ray image 28, because the dose of the X-ray is low.

Then, in order to avoid presence of X-ray images different in image quality as being mixed in X-ray moving images, image processing unit 23 performs image processing for modifying second X-ray image 29 for improving the image quality thereof. Specifically, image processing unit 23 performs third processing for improving the quality of second X-ray image 29 generated in the second processing to quality approximately as high as the quality of first X-ray image 28 by using a trained model trained by machine learning. An X-ray image (a second X-ray image 29A) improved in quality by the third processing is not necessarily exactly identical to an original X-ray image (a first X-ray image generated when the first processing is performed instead of the second processing). In the first embodiment, a trained model trained by deep learning is used.

Machine learning refers to an approach to repetitive training based on given information (for example, a training data set) for autonomous establishment of rules or criteria. Deep learning refers to machine learning using a neural network in a multilayered structure.

The trained model is generated, for example, by repeatedly performing training processing by using a training data set. The training data set includes, for example, a plurality of pieces of training data obtained by labeling a low-quality image given as input with a high-quality image given as output. The training data can be prepared, for example, by lowering the quality of a high-quality image. The trained model trained with the training data set as above enhances the quality of an input image and provides the resultant image.

With improvement in quality of second X-ray image 29 through the third processing, even though the first processing and the second processing are alternately performed at the prescribed time interval, X-ray moving images as high in quality as in the example where the first processing is performed at the prescribed time interval can be taken. The first processing and the second processing alternately performed at the prescribed time interval are by way of example, and a ratio between the first processing performed and the second processing performed should only appropriately be set depending on contents of a therapy or an examination in which X-ray imaging system 100 according to the first embodiment is used. By changing some of the first processing performed at the prescribed time interval to the second processing and performing the third processing, dosage of subject 50 can be lowered while lowering in moving image quality of X-ray moving images is suppressed.

### Processing Performed in Imaging Apparatus and Image Processing Apparatus

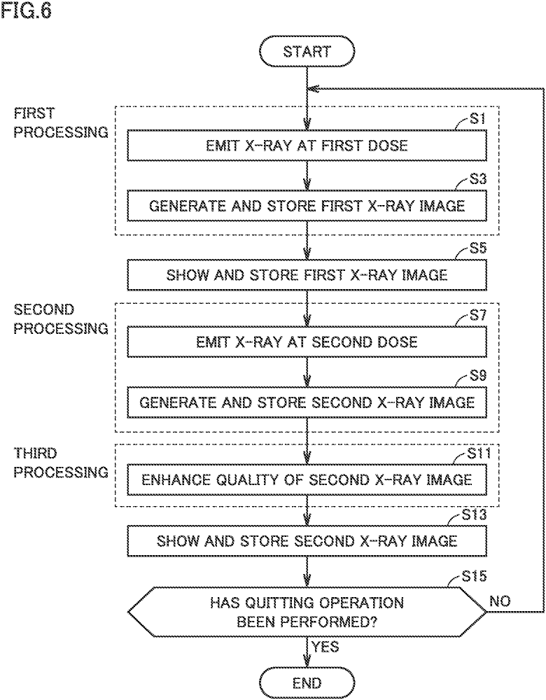

FIG. 6 is a flowchart showing an exemplary processing procedure performed in imaging apparatus 10 and image processing apparatus 20 according to the first embodiment. Processing shown in this flowchart is started, for example, when a user performs an operation to start X-ray imaging through operation unit 8.

As the processing shown in the flowchart is started, the first processing and the second processing are alternately performed at the prescribed time interval until the user performs an operation to quit X-ray imaging through operation unit 8. Initially, in step (the step being abbreviated as “S” below) 1 and S3, imaging apparatus 10 and image processing apparatus 20 perform the first processing. Specifically, in S1, imaging apparatus 10 emits the X-ray to subject 50 at the first dose from X-ray emitter 1. X-ray detector 2 detects the X-ray that has passed through subject 50 and imaging table 3 and provides a detection signal to image processing apparatus 20.

In S3, image generator 22 of image processing apparatus 20 generates first X-ray image 28 based on a detection signal obtained from X-ray detector 2. Then, image generator 22 provides image data of generated first X-ray image 28 to imaging apparatus 10. Image generator 22 may provide generated first X-ray image 28 to imaging apparatus 10 and may have first X-ray image 28 stored in storage 25.

In S5, based on the obtained image data, imaging apparatus 10 has first X-ray image 28 shown on display 7 and stored in storage 9.

As a prescribed time period has elapsed since the first processing was performed, in S7 and S9, imaging apparatus 10 and image processing apparatus 20 perform the second processing. Specifically, in S7, imaging apparatus 10 emits the X-ray to subject 50 at the second dose lower than the first dose from X-ray emitter 1. X-ray detector 2 detects the X-ray that has passed through subject 50 and imaging table 3 and provides a detection signal to image processing apparatus 20.

In S9, image generator 22 of image processing apparatus 20 generates second X-ray image 29 based on a detection signal obtained from X-ray detector 2. Then, image generator 22 provides generated second X-ray image 29 to image processing unit 23.

Then, in S11, image processing apparatus 20 performs the third processing to improve the quality of second X-ray image 29 generated in S9. Specifically, image processing unit 23 inputs second X-ray image 29 generated by image generator 22 in S9 into the trained model and generates second X-ray image 29A resulting from enhancement of the quality of second X-ray image 29. Then, image processing unit 23 provides second X-ray image 29A enhanced in quality to imaging apparatus 10. Image processing unit 23 may provide second X-ray image 29A enhanced in quality to imaging apparatus 10 and may have second X-ray image 29A enhanced in quality stored in storage 25.

In S13, based on the obtained image data, imaging apparatus 10 has second X-ray image 29A shown on display 7 and stored in storage 9.

In S15, imaging apparatus 10 determines whether or not an operation to quit X-ray imaging has been performed. Determination as to whether or not the operation to quit X-ray imaging has been performed may be made at timing of switching between the first processing and the second processing (that is, between S5 and S7) instead of or in addition to S15.

When the operation to quit the X-ray imaging has not been performed (NO in S15), imaging apparatus 10 has the process return to S1 and continues X-ray imaging. In other words, the first processing and the second processing are continued.

When the operation to quit the X-ray imaging has been performed (YES in S15), imaging apparatus 10 quits the process and quits X-ray imaging.

As set forth above, in the first embodiment, in X-ray imaging system 100 that generates an X-ray image by irradiating subject 50 with the X-ray at a prescribed time interval, the dose of the X-ray emitted to subject 50 is lowered every other time. In other words, the first processing and the second processing are alternately performed at the prescribed time interval. Second X-ray image 29 generated with the dose of the X-ray being lowered is lower in quality than first X-ray image 28. Therefore, the quality of second X-ray image 29 is improved by performing the third processing.

By alternately performing the first processing and the second processing at the prescribed time interval as above, dosage of subject 50 can be lower than in the example where the first processing is performed at the prescribed time interval. Though second X-ray image 29 generated in the second processing is lower in quality than first X-ray image 28 generated in the first processing, it is enhanced in quality to be second X-ray image 29A as high in quality as first X-ray image 28 through the third processing. Therefore, even when the first processing and the second processing are alternately performed at the prescribed time interval, X-ray moving images approximately as high in moving image quality as those in the example where the first processing is performed at the prescribed time interval can be taken. In other words, X-ray imaging system 100 according to the first embodiment can achieve lowering in dosage of subject 50 while lowering in moving image quality of X-ray moving images is suppressed.

### First Modification

In the first embodiment, image processing apparatus 20 successively provides generated X-ray images (first X-ray image 28 and second X-ray image 29A) to imaging apparatus 10. Then, imaging apparatus 10 has the obtained X-ray images successively shown on display 7. In other words, in the first embodiment, X-ray images generated at the prescribed time interval are shown in real time. In order to show X-ray images in real time, image processing apparatus 20 enhances the quality of second X-ray image 29 each time second X-ray image 29 is generated. When representation of X-ray images in real time is not required, however, stored second X-ray images 29 may collectively be enhanced in quality after the end of X-ray imaging. In a first modification, a configuration for collectively enhancing the quality of stored second X-ray images 29 after the end of X-ray imaging will be described.

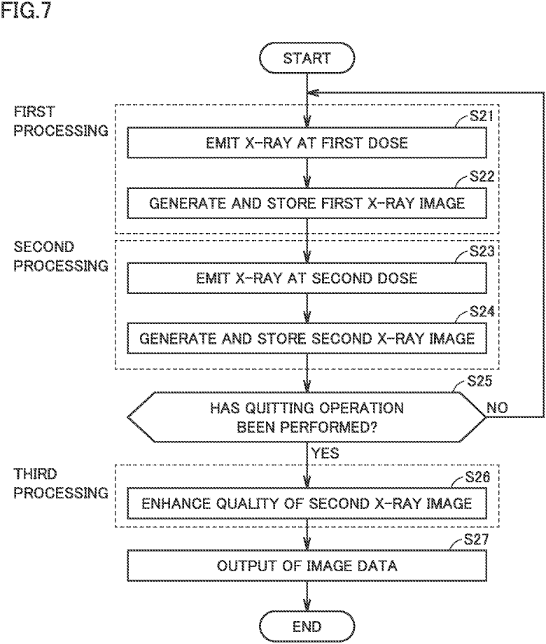

FIG. 7 is a flowchart showing an exemplary processing procedure performed in imaging apparatus 10 and image processing apparatus 20 according to the first modification. Processing shown in this flowchart is started, for example, when a user performs an operation to start X-ray imaging through operation unit 8. As the processing shown in the flowchart is started, the first processing and the second processing are alternately performed at the prescribed time interval until the user performs an operation to quit X-ray imaging through operation unit 8.

In S21 and S22, imaging apparatus 10 and image processing apparatus 20 perform the first processing. Specifically, in S21, imaging apparatus 10 emits the X-ray to subject 50 at the first dose from X-ray emitter 1. X-ray detector 2 detects the X-ray that has passed through subject 50 and imaging table 3 and provides a detection signal to image processing apparatus 20.

In S22, image generator 22 of image processing apparatus 20 generates first X-ray image 28 based on a detection signal obtained from X-ray detector 2. Then, image generator 22 has generated first X-ray image 28 stored in storage 25.

As a prescribed time period has elapsed since the first processing was performed, imaging apparatus 10 and image processing apparatus 20 perform the second processing. Specifically, in S23, imaging apparatus 10 emits the X-ray to subject 50 at the second dose lower than the first dose from X-ray emitter 1. X-ray detector 2 detects the X-ray that has passed through subject 50 and imaging table 3 and provides a detection signal to image processing apparatus 20.

In S24, image generator 22 generates second X-ray image 29 based on a detection signal obtained from X-ray detector 2. Then, image generator 22 has generated second X-ray image 29 stored in storage 25.

In S25, imaging apparatus 10 determines whether or not an operation to quit X-ray imaging has been performed. Determination as to whether or not the operation to quit X-ray imaging has been performed may be made at timing of switching between the first processing and the second processing instead of or in addition to S25.

When the operation to quit the X-ray imaging has not been performed (NO in S25), imaging apparatus 10 has the process return to S21 and continues X-ray imaging. In other words, the first processing and the second processing are continued.

When the operation to quit the X-ray imaging has been performed (YES in S25), imaging apparatus 10 quits X-ray imaging and has the process proceed to S26.

In S26, image processing apparatus 20 performs the third processing to improve the quality of second X-ray image 29 stored in storage 25. Specifically, image processing unit 23 inputs second X-ray image 29 stored in storage 25 into the trained model and generates second X-ray image 29A resulting from enhancement of the quality of second X-ray image 29. Then, image processing unit 23 updates second X-ray image 29 stored in storage 25 to second X-ray image 29A.

In S27, image processing apparatus 20 provides image data 27 of first X-ray image 28 and second X-ray image 29A generated by performing the processing shown in the present flowchart to imaging apparatus 10. For example, imaging apparatus 10 has image data 27 obtained from image processing apparatus 20 shown on display 7 and stored in storage 9. Image processing apparatus 20 may erase image data 27 stored in storage 25 after it provides image data 27 to imaging apparatus 10.

As set forth above, dosage of subject 50 can be lowered while lowering in moving image quality of X-ray moving images is suppressed as in the first embodiment, also by performing the third processing to collectively enhance the quality of second X-ray images 29 after the end of X-ray imaging.

### Second Modification

In the first embodiment, an example in which the first processing and the second processing are alternately performed at the prescribed time interval is described. The first processing and the second processing are not limited to those being alternately performed. For example, after the first processing is performed, the second processing may be performed a plurality of times. A ratio between the first processing and the second processing to be performed can appropriately be set depending on contents of a therapy or an examination in which X-ray imaging system 100 is used. In a second modification, an example in which the second processing is performed two times after the first processing is performed will be described.

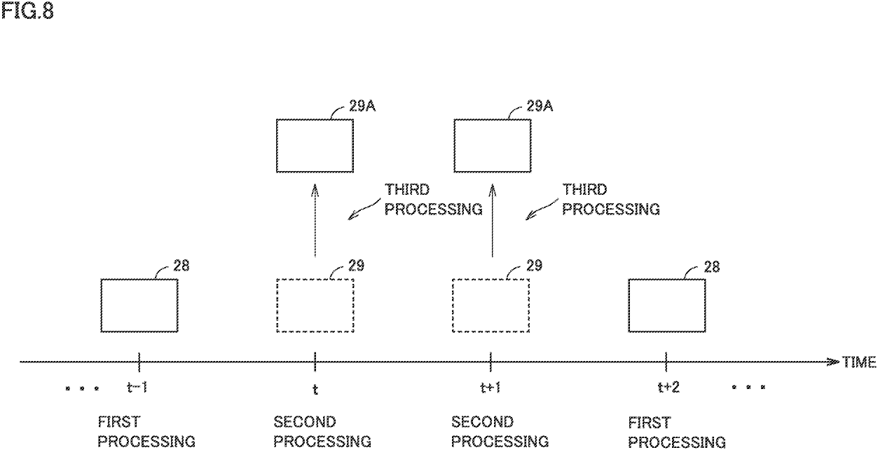

FIG. 8 is a schematic diagram for illustrating taking of X-ray moving images according to the second modification. FIG. 8 shows irradiation with the X-ray at a prescribed time interval (..., t-1, t, t+1, t+2, ...) and generation of an X-ray image at each time point.

Referring to FIG. 8, at time t-1 and time t+2, the first processing for generating first X-ray image 28 is performed. At time t and time t+1, the second processing for generating second X-ray image 29 is performed.

Then, as in the first embodiment, the third processing is performed on second X-ray image 29 to enhance the quality thereof to obtain second X-ray image 29A approximately as high in quality as first X-ray image 28.

As set forth above, by performing the second processing a plurality of times (two times in the example above) after the first processing was performed, dosage of subject 50 can be lower than in the example where the first processing is performed at the prescribed time interval.

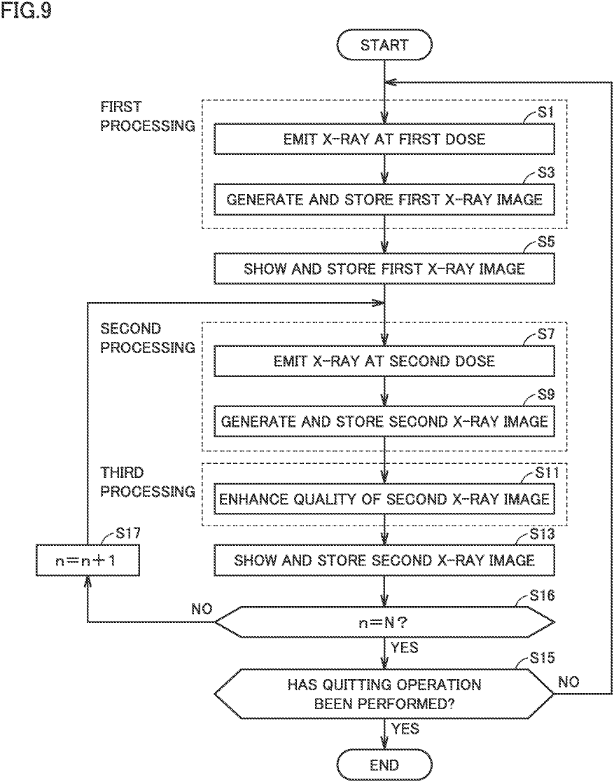

FIG. 9 is a flowchart showing an exemplary processing procedure performed in imaging apparatus 10 and image processing apparatus 20 according to the second modification. Processing shown in this flowchart is different from the processing in the flowchart in FIG. 6 in addition of S16 and S17. Since the processing is otherwise similar to the processing in the flowchart in FIG. 6, the same reference characters as those in the flowchart in FIG. 6 are allotted and description will not be repeated.

In S16, imaging apparatus 10 determines whether or not the number of times n of the second processing performed since the first processing was performed has reached the number of times N set in advance. The number of times N according to the second modification is set to two. Specifically, in S16, imaging apparatus 10 determines whether or not the second processing have been performed two times since the first processing was performed.

When the number of times n of the second processing performed has not reached the number of times N (NO in S16), imaging apparatus 10 has the process proceed to S17. In S17, imaging apparatus 10 adds one to the number of times n of the second processing performed, and has the process return to S7. Then, imaging apparatus 10 performs the second processing again.

When the number of times n of the second processing performed has reached the number of times N (YES in S16), imaging apparatus 10 has the process proceed to S15. In this case, imaging apparatus 10 clears the number of times n of the second processing performed.

As set forth above, also in the second modification, dosage of subject 50 can be lower than in the example in which the first processing is performed at the prescribed time interval. As the number of times N is increased, that is, as the number of times of the second processing performed increases, dosage of subject 50 can be lowered.

The second modification can also be combined with the first modification described above. Dosage of subject 50 can be lower than in the example in which the first processing is performed at the prescribed time interval also by combination with the first modification.

### Third Modification

In the first embodiment and the first modification, an example in which second X-ray image 29A enhanced in quality is used as one X-ray image in X-ray moving images is described. In other words, second X-ray image 29A enhanced in quality is used as one frame of X-ray moving images. Second X-ray image 29A enhanced in quality may be used in another application. In a third modification, an example in which second X-ray image 29A enhanced in quality is used in creation of a superimposed image for improving viewability of stent 31 in an X-ray image will be described.

Stent 31 is small in difference in X-ray transmittance from body tissues and blood vessels of subject 50. Therefore, in an X-ray image, viewability of stent 31 may be low. Then, by superimposing (integrating) a plurality of X-ray images after alignment based on the positions of markers 34 and 35, the superimposed image can be created. Specifically, image processing unit 23 selects an X-ray image to be a reference (which is also referred to as a “reference image” below) at prescribed timing from among first X-ray images 28 generated by image generator 22. Then, image processing unit 23 superimposes first X-ray image 28 in a frame other than the reference image and second X-ray image 29A enhanced in quality on the reference image after alignment. As the superimposed image is created and shown on display 7, viewability of stent 31 can be improved.

Images are aligned by moving, zooming in or out, or rotating the images such that markers 34 and 35 in the images are superimposed with the positions of markers 34 and 35 in the reference image being defined as the reference.

Second X-ray image 29A enhanced in quality can also be used for creation of the superimposed image as above. In this case as well, dosage of subject 50 can be lowered.

### Second Embodiment

Another approach to lowering in dosage of subject 50 while lowering in moving image quality of X-ray moving images is suppressed will be described. When real-time representation of an X-ray image is not required as in the first modification described above, an X-ray imaging system 101 (see FIG. 1) according to a second embodiment can be applied. In X-ray imaging system 101 according to the second embodiment, a time interval (a prescribed time interval) for irradiation of subject 50 with the X-ray is made longer. Dosage of subject 50 can thus be lowered. In this case, however, there is a concern about a lower frame rate, a longer interval between frames, and missing of information between frames. Then, in X-ray imaging system 101 according to the second embodiment, the prescribed time interval is made longer and an X-ray image between frames is generated by using successive X-ray images generated at a prescribed time interval.

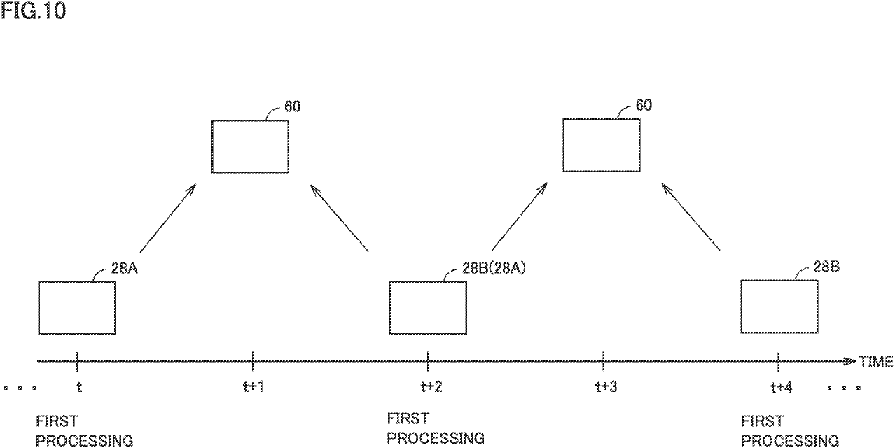

FIG. 10 is a schematic diagram for illustrating taking of X-ray moving images according to the second embodiment. FIG. 10 shows irradiation with the X-ray at a prescribed time interval (..., t, t+2, t+4, ...) and generation of an X-ray image at each time point. Specifically, at time t, time t+2, and time t+4, the first processing for generating first X-ray image 28 of subject 50 by irradiating subject 50 with the X-ray at the first dose is performed.

The prescribed time interval according to the second embodiment is, for example, two times as long as the time interval according to the first embodiment. Therefore, an amount of irradiation with the X-ray is half that in the first processing performed at the prescribed time interval according to the first embodiment, and hence dosage of subject 50 can be lowered. On the other hand, an interval between frames of X-ray moving images is longer, and information between frames is missed and moving image quality of the X-ray moving images is lower.

Then, an X-ray image (which is also referred to as an “intermediate image” below) 60 at time (for example, time t+1) between time t and time t+2 is generated from a first X-ray image 28A generated at time t and a first X-ray image 28B generated at time t+2, based on the trained model trained by machine learning. More specifically, in the second embodiment, the trained model trained by deep learning is used. This is also applicable to intermediate image 60 at time t+3 between time t+2 and time t+4.

The trained model is generated, for example, by repeatedly performing training processing by using a training data set. The training data set includes, for example, a plurality of pieces of training data obtained by labeling two successive images given as input with an image corresponding to a temporally intermediate image between the two images that is given as output. Training data can be prepared, for example, by adopting first and third images among three successively generated images as an image to be given as input and by adopting the second image as an image to be given as output. The trained model trained with the training data set as above generates an image temporally intermediate between two input images and provides the intermediate image.

By generating intermediate image 60 at time t+1 between time t and time t+2, information missed between frames can be compensated for even when the prescribed time interval is made longer. Therefore, lowering in moving image quality of X-ray moving images can be suppressed. First X-ray image 28A generated at time t corresponds to an exemplary “third X-ray image” according to this invention. First X-ray image 28B generated at time t+2 corresponds to an exemplary “fourth X-ray image” according to this invention. The prescribed time interval should only appropriately be set depending on contents of a therapy or an examination in which X-ray imaging system 101 according to the second embodiment is used.

### Overall Configuration According to Second Embodiment

Referring again to FIG. 1, X-ray imaging system 101 according to the second embodiment includes an image processing apparatus 210 instead of image processing apparatus 20 in the first embodiment. Since the configuration is otherwise similar to that in the first embodiment, description will not be repeated.

Image processing apparatus 210 is different in that image processing unit 23 in the first embodiment is replaced with an image processing unit 231. First X-ray image 28 and intermediate image 60 described above are stored as image data 27 in storage 25.

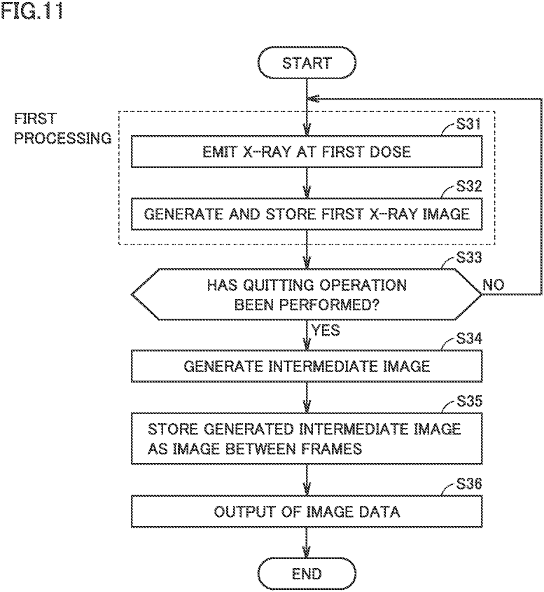

FIG. 11 is a flowchart showing an exemplary processing procedure performed in imaging apparatus 10 and image processing apparatus 210 according to the second embodiment. Processing shown in this flowchart is started, for example, when a user performs an operation to start X-ray imaging through operation unit 8.

As the processing shown in the flowchart is started, the first processing is performed at the prescribed time interval until the user performs an operation to quit X-ray imaging through operation unit 8. Specifically, in S31, imaging apparatus 10 emits the X-ray to subject 50 at the first dose from X-ray emitter 1. X-ray detector 2 detects the X-ray that has passed through subject 50 and imaging table 3 and provides a detection signal to image processing apparatus 210.

In S32, image generator 22 of image processing apparatus 210 generates first X-ray image 28 based on a detection signal obtained from X-ray detector 2. Then, image generator 22 has generated first X-ray image 28 stored in storage 25.

In S33, imaging apparatus 10 determines whether or not an operation to quit X-ray imaging has been performed. When the operation to quit the X-ray imaging has not been performed (NO in S33), imaging apparatus 10 has the process return to S31 and continues X-ray imaging. In other words, the first processing is performed at the prescribed time interval.

When the operation to quit the X-ray imaging has been performed (YES in S33), imaging apparatus 10 quits X-ray imaging and has the process proceed to S34.

In S34, image processing apparatus 210 generates intermediate image 60 by using first X-ray images 28 stored in storage 25. Specifically, image processing unit 231 of image processing apparatus 210 reads successive first X-ray images 28 from storage 25. An X-ray image generated earlier among successive first X-ray images 28 corresponds to first X-ray image 28A described above, and an X-ray image generated later corresponds to first X-ray image 28B described above. By way of example, it is assumed that first X-ray image 28 generated at time t is read as first X-ray image 28A and first X-ray image 28 generated at time t+2 is read as first X-ray image 28B. Image processing unit 231 generates intermediate image 60 at time t+1 between time t and time t+2 by inputting first X-ray image 28A and first X-ray image 28B which are successive first X-ray images 28 into the trained model. For example, when the operation to quit the process is performed after the first processing is performed once, intermediate image 60 is not generated in S34.

In S35, image processing apparatus 210 has generated intermediate image 60 stored in storage 25 as the X-ray image at time t+1. In other words, image processing unit 231 has intermediate image 60 stored in storage 25 as an image between frames.

In S36, image processing apparatus 210 provides image data 27 stored in storage 25, that is, the X-ray images (first X-ray image 28 and intermediate image 60) generated in S34 and S35, to imaging apparatus 10. For example, imaging apparatus 10 has image data obtained from image processing apparatus 210 shown on display 7 and stored in storage 9. Image processing apparatus 210 may erase image data 27 stored in storage 25 after it provides the image data to imaging apparatus 10.

As set forth above, X-ray imaging system 101 according to the second embodiment lowers dosage of subject 50 by increasing the time interval (the prescribed time interval) for irradiation of subject 50 with the X-ray. Then, by generating the intermediate image between frames, missed information between frames is compensated for. Lowering in moving image quality of X-ray moving images can thus be suppressed. In other words, X-ray imaging system 101 according to the second embodiment can achieve lowering in dosage of subject 50 while lowering in moving image quality of X-ray moving images is suppressed.

### Fourth Modification

In the second embodiment, though intermediate image 60 is generated by using the trained model trained by machine learning, intermediate image 60 should only be generated and means for generating intermediate image 60 is not limited to use of the trained model trained by machine learning. In a fourth modification, a configuration for generating intermediate image 60 by interpolation processing such as linear interpolation will be described.

In the fourth modification, by way of example, an example in which intermediate image 60 is generated by linear interpolation by using successive first X-ray image 28A and first X-ray image 28B will be described. It is assumed that first X-ray image 28A is a first X-ray image generated at time t in FIG. 10 and first X-ray image 28B is a first X-ray image generated at time t+2.

Pixel values of corresponding pixels in first X-ray image 28A and first X-ray image 28B are compared with each other. A difference between the pixel value of first X-ray image 28A and the pixel value of first X-ray image 28B in the corresponding pixels can be concluded as an amount of change in pixel value of the pixels from time t to time t+2. Then, for example, the pixel value at time t+1 of one pixel can be a value intermediate between the pixel value of first X-ray image 28A and the pixel value of first X-ray image 28B. A pixel the value of which is not varied between first X-ray image 28A and first X-ray image 28B can have a value equal to the value of first X-ray image 28A and first X-ray image 28B.

By performing the interpolation processing above for all pixels by using first X-ray image 28A and first X-ray image 28B, intermediate image 60 at time t+1 can be generated.

An effect similar to the effect in the second embodiment can be achieved also by generating the intermediate image by the interpolation processing, instead of using the trained model trained by machine learning.

### Fifth Modification

In the second embodiment, an example in which a single intermediate image which is an X-ray image between frames is generated by increasing the time interval (the prescribed time interval) for irradiation of subject 50 with the X-ray and by using successive X-ray images generated at the prescribed time interval is described. The number of generated intermediate images, however, is not limited to one. A plurality of intermediate images may be generated by using successive X-ray images generated at the prescribed time interval. In a fifth modification, an example in which a plurality of intermediate images are generated by using successive X-ray images generated at the prescribed time interval will be described.

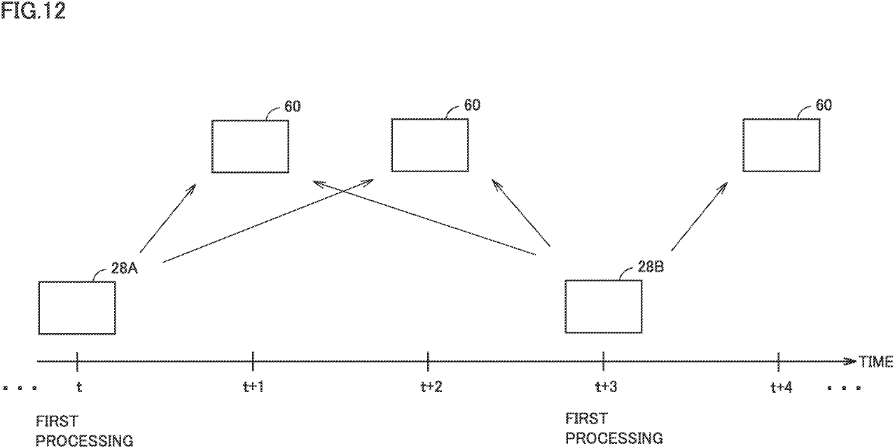

FIG. 12 is a schematic diagram for illustrating taking of X-ray moving images according to the fifth modification. FIG. 12 shows irradiation with the X-ray at a prescribed time interval (..., t, t+3, ...) and generation of an X-ray image at each time point. Specifically, at time t and time t+3, the first processing for generating first X-ray image 28 of subject 50 by irradiating subject 50 with the X-ray at the first dose is performed.

The prescribed time interval according to the fifth modification is, for example, three times as long as the time interval according to the first embodiment. Therefore, an amount of irradiation with the X-ray is one third that in the first processing performed at the prescribed time interval according to the first embodiment, and hence dosage of subject 50 can be lower.

Image processing apparatus 210 generates intermediate images at time t+1 and time t+2 which are times between time t and time t+3 by using the trained model trained by machine learning.

The trained model is generated, for example, by repeatedly performing training processing by using a training data set. The training data set includes, for example, a plurality of pieces of training data obtained by labeling two successive images given as input with two images (the two images being images different in time) corresponding to temporally intermediate images between the two images, that are given as output. Specifically, the training data can be prepared, for example, by adopting first and fourth images among four successively generated images as images to be given as input and by adopting second and third images as images to be given as output. The trained model trained with the training data set as above generates two images temporally intermediate between two input images and provides the intermediate images.

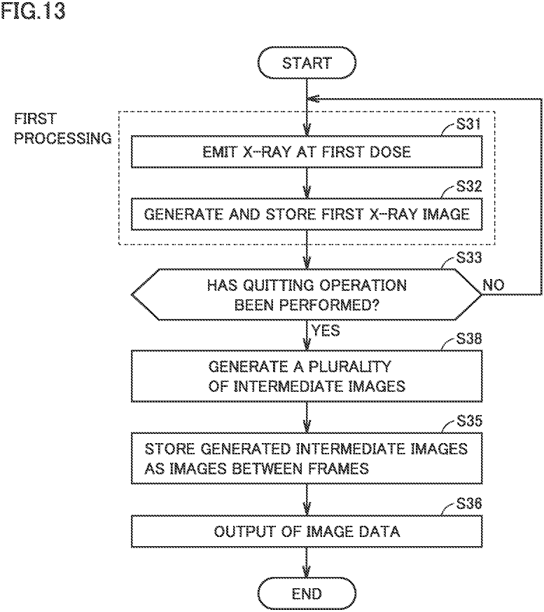

FIG. 13 is a flowchart showing an exemplary processing procedure performed in imaging apparatus 10 and image processing apparatus 210 according to the fifth modification.

Processing shown in this flowchart is different from the processing in the flowchart in FIG. 11 in that S34 is replaced with S38. Since the processing is otherwise similar to the processing in the flowchart in FIG. 11, the same reference characters as those in the flowchart in FIG. 11 are allotted and description will not be repeated.

In S38, image processing apparatus 210 generates a plurality of intermediate images 60 by using first X-ray images 28 stored in storage 25. Specifically, it is assumed that first X-ray image 28 generated at time t is read as first X-ray image 28A and first X-ray image 28 generated at time t+3 is read as first X-ray image 28B. Image processing unit 231 of image processing apparatus 210 generates intermediate images 60 at time t+1 and time t+2 between time t and time t+3 by inputting first X-ray image 28A and first X-ray image 28B which are successive first X-ray images 28 into the trained model.

In following S35, intermediate images 60 generated in S38 are stored in storage 25 as the images between frames.

By generating a plurality of intermediate images by using successive X-ray images generated at the prescribed time interval as above, the prescribed time interval for irradiation of subject 50 with the X-ray can be increased while the frame rate is maintained. Thus, dosage of subject 50 can be lowered. Alternatively, by generating a plurality of intermediate images by using successive X-ray images generated at the prescribed time interval, the frame rate of X-ray moving images can also be increased while the prescribed time interval for irradiation of subject 50 with the X-ray is maintained.

### Third Embodiment

Yet another approach to lowering in dosage of subject 50 while lowering in moving image quality of X-ray moving images is suppressed will be described. In a third embodiment, an example for generating an X-ray image 70 to be shown in a next frame (which is also referred to as a “prediction image” below) by using X-ray images generated in the past will be described. In an X-ray imaging system 102 (see FIG. 1) according to the third embodiment, a prediction image can be generated from X-ray images in the past and hence an X-ray image can be shown in real time.

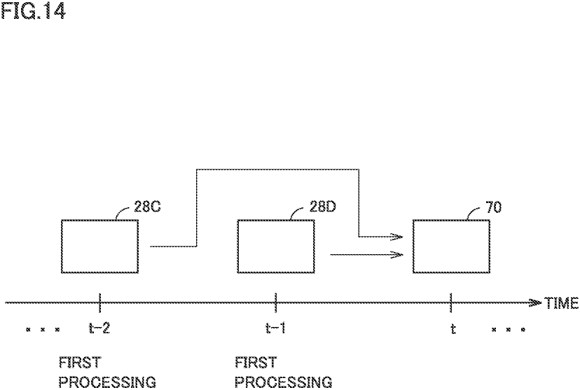

FIG. 14 is a schematic diagram for illustrating taking of X-ray moving images according to the third embodiment. In FIG. 14, by way of example, in X-ray imaging system 102 that generates an X-ray image by irradiating subject 50 with the X-ray at a prescribed time interval (..., t-2, t-1, ...), irradiation of subject 50 with the X-ray is skipped at a frequency of once in two times at the prescribed time interval and a prediction image 70 is generated by using X-ray images 28 generated in the past. Specifically, at time t-2 and time t-1, subject 50 is irradiated with the X-ray at the first dose to generate first X-ray images 28C and 28D. Then, at time t, irradiation of subject 50 with the X-ray is skipped. Then, prediction image 70 at time t is generated by inputting first X-ray images 28C and 28D which are X-ray images generated in the past into the trained model trained by machine learning. In the third embodiment, the trained model trained by deep learning is used.

The trained model is generated, for example, by repeatedly performing training processing by using a training data set. The training data set includes, for example, a plurality of pieces of training data obtained by labeling images in the past given as input with an image temporally successive to the images in the past, that is given as output. The training data can be prepared, for example, by adopting an image generated earlier as an image given as input and adopting an image generated later as an image given as output, among temporally successively generated images. A plurality of temporally successive images may be employed as images given as input. The trained model trained with the training data set as above provides an image resulting from prediction of a next frame from the inputted images.

For example, irradiation of subject 50 with the X-ray at time t is skipped, and prediction image 70 at time t is generated by inputting first X-ray image 28C generated at time t-2 and first X-ray image 28D generated at time t-1 into the trained model. By skipping irradiation of subject 50 with the X-ray at time t, dosage of subject 50 in taking X-ray moving images can be lowered. Then, by generating prediction image 70, lowering in moving image quality of X-ray moving images can be suppressed without lowering in frame rate of the X-ray moving images. An interval at which irradiation of subject 50 with the X-ray is skipped should only be set appropriately depending on contents of a therapy or an examination in which X-ray imaging system 102 according to the third embodiment is used. In the third embodiment, after irradiation with the X-ray is carried out two times, irradiation with the X-ray is skipped when next prescribed time comes.

### Overall Configuration According to Third Embodiment

Referring again to FIG. 1, X-ray imaging system 102 according to the third embodiment includes an image processing apparatus 220 instead of image processing apparatus 20 in the first embodiment. Since the configuration is otherwise similar to that of the first embodiment, description will not be repeated.

Image processing apparatus 220 is different in that image processing unit 23 in the first embodiment is replaced with an image processing unit 232.

When image generator 22 generates first X-ray image 28, it provides image data of first X-ray image 28 to imaging apparatus 10 and to image processing unit 232. When image processing unit 232 obtains two first X-ray images 28 from image generator 22, it generates prediction image 70. In other words, image processing unit 232 accepts first X-ray images 28C and 28D in two successive frames in the past as input, and generates prediction image 70 in the next frame. Image processing unit 232 provides image data of generated prediction image 70 to imaging apparatus 10.

Based on the obtained image data, imaging apparatus 10 has first X-ray image 28 and prediction image 70 shown on display 7 and stored in storage 9.

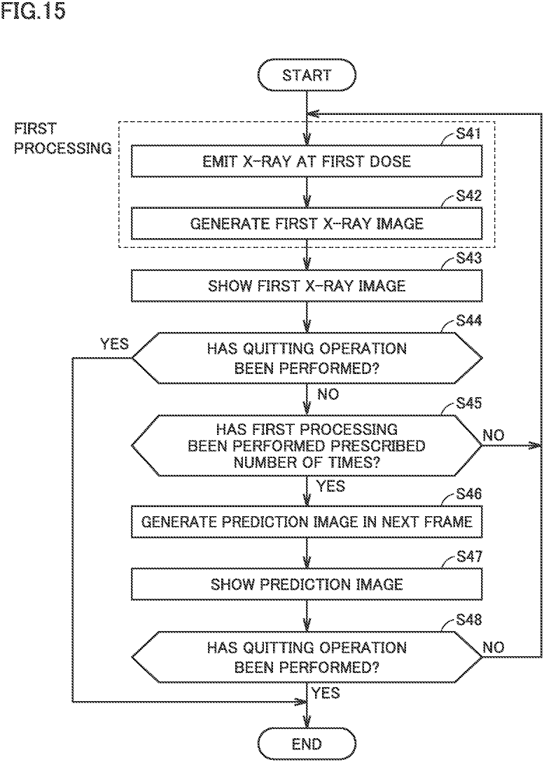

FIG. 15 is a flowchart showing an exemplary processing procedure performed in imaging apparatus 10 and image processing apparatus 220 according to the third embodiment. Processing shown in this flowchart is started, for example, when a user performs an operation to start X-ray imaging through operation unit 8.

As the processing shown in the flowchart is started, imaging apparatus 10 and image processing apparatus 220 perform the first processing and processing for generating prediction image 70 at the prescribed time interval until the user performs an operation to quit X-ray imaging through operation unit 8. Specifically, in S41, imaging apparatus 10 emits the X-ray to subject 50 at the first dose from X-ray emitter 1. X-ray detector 2 detects the X-ray that has passed through subject 50 and imaging table 3 and provides a detection signal to image processing apparatus 220.

In S42, image generator 22 of image processing apparatus 220 generates first X-ray image 28 based on a detection signal obtained from X-ray detector 2. Then, image generator 22 provides image data of generated first X-ray image 28 to imaging apparatus 10 and to image processing unit 232.

In S43, based on the obtained image data, imaging apparatus 10 has first X-ray image 28 shown on display 7 and stored in storage 9.

In S44, imaging apparatus 10 determines whether or not an operation to quit X-ray imaging has been performed. When the operation to quit the X-ray imaging has been performed (YES in S44), imaging apparatus 10 quits the process. When the operation to quit the X-ray imaging has not been performed (NO in S44), imaging apparatus 10 has the process proceed to S45.

In S45, imaging apparatus 10 determines whether or not it has performed the first processing a prescribed number of times. The prescribed number of times can be set depending on contents of a therapy or an examination conducted with the use of X-ray imaging system 102. The prescribed number of times corresponds to the number of first X-ray images to be used for generating prediction image 70. In other words, since first X-ray image 28 is generated each time the first processing is performed, prediction image 70 is generated by using generated first X-ray image 28. In the example in FIG. 14 described above, the prescribed number of times is set to two.

When the first processing has not been performed the prescribed number of times (NO in S45), imaging apparatus 10 increments the prescribed number of times, has the process return to S41, and performs the first processing again. When the first processing has been performed the prescribed number of times (YES in S45), imaging apparatus 10 has the process proceed to S46.

In S46, image processing apparatus 220 generates prediction image 70 in the next frame by using first X-ray image 28 generated by performing the first processing. Specifically, image processing unit 232 generates prediction image 70 by inputting first X-ray image 28 generated by performing the first processing into the trained model. Image processing unit 232 provides image data of generated prediction image 70 to imaging apparatus 10.

In S47, based on the obtained image data, imaging apparatus 10 has prediction image 70 shown on display 7 and stored in storage 9.

In S48, imaging apparatus 10 determines whether or not an operation to quit X-ray imaging has been performed. When the operation to quit the X-ray imaging has been performed (YES in S48), imaging apparatus 10 quits the process. When the operation to quit the X-ray imaging has not been performed (NO in S48), imaging apparatus 10 resets count of the prescribed number of times and has the process return to S41. In a next loop, prediction image 70 is generated by using first X-ray images 28 generated until the number of times of processing reaches the prescribed number of times since the count has been reset.

As set forth above, in the third embodiment, in X-ray imaging system 102 that generates an X-ray image by irradiating subject 50 with the X-ray at the prescribed time interval, irradiation of subject 50 with the X-ray is skipped at the frequency of once in two times at the prescribed time interval. When irradiation with the X-ray is skipped, no first X-ray image can be generated, however, prediction image 70 is generated instead of the first X-ray image. In other words, by skipping irradiation with the X-ray every prescribed number of times, dosage of subject 50 is lowered. Then, an image in a frame missed by skipping irradiation with the X-ray is generated as prediction image 70 by using X-ray images in the past. Lowering in moving image quality of X-ray moving images can thus be suppressed. In other words, X-ray imaging system 102 according to the third embodiment can lower dosage of subject 50 without lowering in moving image quality of X-ray moving images.

Interpolation processing described in the fourth modification above can be applied also in the third embodiment. In other words, for example, prediction image 70 may be generated by interpolation processing, instead of the trained model trained by machine learning. In this case, prediction image 70 can be generated, for example, by linear interpolation using first X-ray images 28C and 28D.

### Aspects

Illustrative embodiments described above are understood by a person skilled in the art as specific examples of aspects below.

(Clause 1) An X-ray imaging method according to one aspect is an X-ray imaging method of taking an X-ray image of a subject, and includes irradiating the subject with an X-ray at a first dose and taking a first X-ray image of the subject, irradiating the subject with an X-ray at a second dose lower than the first dose and taking a second X-ray image of the subject, and inputting the second X-ray image into a trained model trained by machine learning to modify the second X-ray image.

According to the X-ray imaging method described in Clause 1, the dosage of the subject can be lowered while lowering in quality of the X-ray image is suppressed.

(Clause 2) In the X-ray imaging method described in Clause 1, the trained model is generated by training processing using a training data set. The training data set includes a plurality of pieces of training data obtained by labeling an image given as input to the machine learning with an image, that is given as output from the machine learning, higher in quality than the image given as the input.

According to the X-ray imaging method described in Clause 2, image quality of the second X-ray image can appropriately be improved.

(Clause 3) In the X-ray imaging method described in Clause 1 or 2, the taking a second X-ray image is performed after performing the taking a first X-ray image a predetermined prescribed number of times.

According to the X-ray imaging method described in Clause 3, a ratio between the taking a first X-ray image and the taking a second X-ray image can appropriately be set depending on contents of diagnosis or examination in which the X-ray imaging method is used.

(Clause 4) The X-ray imaging method described in Clause 1 further includes showing the taken X-ray image. In the showing a taken X-ray image, the first X-ray image and the modified second X-ray image are shown at different times.

According to the X-ray imaging method described in Clause 4, since the first X-ray image and the modified second X-ray image are shown at different times, the X-ray images can be shown in a format of moving images.

(Clause 5) The X-ray imaging method described in Clause 1 further includes integrating the first X-ray image and the modified second X-ray image.

According to the X-ray imaging method described in Clause 5, by integrating the first X-ray image and the modified second X-ray image with each other, viewability of the X-ray image can be enhanced.

(Clause 6) In the X-ray imaging method described in Clause 5, the integrating the first X-ray image and the modified second X-ray image includes aligning the first X-ray image and the modified second X-ray image with each other.

According to the X-ray imaging method described in Clause 6, since the first X-ray image and the modified second X-ray images are aligned with each other, the first X-ray image and the modified second X-ray image can appropriately be integrated with each other.

(Clause 7) An X-ray imaging method according to one aspect is an X-ray imaging method of taking an X-ray image of a subject, and includes irradiating the subject with an X-ray at a prescribed time interval and taking a third X-ray image and a fourth X-ray image of the subject that are successive, and generating an intermediate image between the third X-ray image and the fourth X-ray image by using the third X-ray image and the fourth X-ray image.

According to the X-ray imaging method described in Clause 7, the dosage of the subject can be lowered while lowering in moving image quality of the X-ray moving images is suppressed.

(Clause 8) In the X-ray imaging method described in Clause 7, in the generating an intermediate image, the intermediate image is generated by inputting the third X-ray image and the fourth X-ray image into a trained model trained by machine learning. The trained model is generated by training processing using a training data set. The training data set includes a plurality of pieces of training data obtained by labeling successive images given as input to the machine learning with an image, that is given as output from the machine learning, corresponding to a temporally intermediate image between the successive images.

According to the X-ray imaging method described in Clause 8, the intermediate image can appropriately be generated from the third X-ray image and the fourth X-ray image by using the trained model.

(Clause 9) In the X-ray imaging method described in Clause 7, in the generating an intermediate image, the intermediate image is generated by interpolation processing using the third X-ray image and the fourth X-ray image.

According to the X-ray imaging method described in Clause 9, the intermediate image can appropriately be generated by interpolation processing using the third X-ray image and the fourth X-ray image.

(Clause 10) An X-ray imaging method according to one aspect is an X-ray imaging method of taking an X-ray image of a subject, and includes irradiating the subject with an X-ray and generating an X-ray image of the subject and generating a prediction image in a next frame of the X-ray image by using the generated X-ray image.

According to the X-ray imaging method described in Clause 10, the dosage of the subject can be lowered while lowering in moving image quality of the X-ray moving images is suppressed.

(Clause 11) In the X-ray imaging method described in Clause 10, in the generating a prediction image, the prediction image is generated by inputting the X-ray image into a trained model trained by machine learning. The trained model is generated by training processing using a training data set. The training data set includes a plurality of pieces of training data obtained by labeling an input image given as input to the machine learning with an image, that is given as output from the machine learning, in the next frame of the input image.

According to the X-ray imaging method described in Clause 11, the prediction image in the next frame can appropriately be generated from the X-ray image by using the trained model.

(Clause 12) In the X-ray imaging method described in Clause 10, in the generating a prediction image, the prediction image is generated by interpolation processing using the X-ray image.

According to the X-ray imaging method described in Clause 12, the prediction image can appropriately be generated by interpolation processing using the X-ray image.

(Clause 13) An X-ray imaging system according to one aspect includes an imaging apparatus configured to successively generate X-ray images of a subject by irradiating the subject with an X-ray and an image processing apparatus that processes the X-ray images. The imaging apparatus is configured to perform processing for irradiating the subject with an X-ray at a first dose and taking a first X-ray image of the subject and processing for irradiating the subject with an X-ray at a second dose lower than the first dose and taking a second X-ray image of the subject. The image processing apparatus is configured to input the second X-ray image into a trained model trained by machine learning to modify the second X-ray image.

(Clause 14) An X-ray imaging system according to one aspect includes an imaging apparatus and an image processing apparatus. The imaging apparatus is configured to take a third X-ray image and a fourth X-ray image of a subject that are successive, by irradiating the subject with an X-ray at a prescribed time interval. The image processing apparatus is configured to generate an intermediate image intermediate between the third X-ray image and the fourth X-ray image by using the third X-ray image and the fourth X-ray image.

(Clause 15) An X-ray imaging system according to one aspect includes an imaging apparatus configured to successively generate X-ray images of a subject by irradiating the subject with an X-ray and an image processing apparatus configured to generate a prediction image in a next frame of the X-ray images by using the generated X-ray images.

According to the X-ray imaging system described in each of Clauses 13 to 15, the dosage of the subject can be lowered while lowering in moving image quality of the X-ray moving images is suppressed.

Combination as appropriate of features described in the embodiments and the modifications above, including combination not mentioned herein, is originally intended so long as no inconvenience or inconsistency is caused.

It should be understood that the embodiments disclosed herein are illustrative and non-restrictive in every respect. The scope of the present disclosure is defined by the terms of the claims rather than the description of the embodiments above, and is intended to include any modifications within the scope and meaning equivalent to the terms of the claims.

### Reference Signs List

1 X-ray emitter; 2 X-ray detector; 3 imaging table; 4 movement mechanism; 5 driver; 6 controller; 7 display; 8 operation unit; 9, 25 storage; 10 imaging apparatus; 20, 210, 220 image processing apparatus; 21 processor; 22 image generator; 23, 231, 232 image processing unit; 27 image data; 28, 28C, 28D first X-ray image; 28A third X-ray image; 28B fourth X-ray image; 29 second X-ray image; 29A second X-ray image (enhanced in quality); 31 stent; 32 guide wire; 33 catheter; 34, 35 marker; 50 subject; 60 intermediate image; 70 prediction image; 100, 101, 102 X-ray imaging system

1. An X-ray imaging method of taking an X-ray image of a subject, the X-ray imaging method comprising:
irradiating the subject with an X-ray at a first dose and taking a first X-ray image of the subject;
irradiating the subject with an X-ray at a second dose lower than the first dose and taking a second X-ray image of the subject; and
inputting the second X-ray image into a trained model trained to improve: quality of the X-ray image by machine learning to modify the second X-ray image.

2. The X-ray imaging method according to claim 1, wherein
the trained model is generated by training processing using a training data set, and
the training data set includes a plurality of pieces of training data obtained by labeling an image given as input to the machine learning with an image, that is given as output from the machine learning, higher in quality than the image given as the input.

3. The X-ray imaging method according to claim 1, wherein
the taking a second X-ray image is performed after performing the taking a first X-ray image a predetermined prescribed number of times.

4. The X-ray imaging method according to claim 1, further comprising showing the taken X-ray image, wherein
in the showing a taken X-ray image, the first X-ray image and the modified second X-ray image are shown at different times.

5. The X-ray imaging method according to claim 1, further comprising integrating the first X-ray image and the modified second X-ray image.

6. The X-ray imaging method according to claim 5, wherein
the integrating the first X-ray image and the modified second X-ray image includes aligning the first X-ray image and the modified second X-ray image with each other.

7-12. (canceled)

13. An X-ray imaging system comprising:
an imaging apparatus configured to successively generate X-ray images of a subject by irradiating the subject with an X-ray; and
an image processing apparatus that processes the X-ray images, wherein
the imaging apparatus is configured to perform (i) processing for irradiating the subject with an X-ray at a first dose and taking a first X-ray image of the subject and (ii) processing for irradiating the subject with an X-ray at a second dose lower than the first dose and taking a second X-ray image of the subject, and
the image processing apparatus is configured to input the second X-ray image into a trained model trained to improve quality of the X-ray images by machine learning to modify the second X-ray image.

14-15. (canceled)
